(19) United States
(12) Patent Application Publication
(10) Pub. No.: US 2022/0134252 A1
(43) Pub. Date: May 5, 2022

(54) ENCLOSED PARTITION DIVIDING WALL DISTILLATION COLUMN AND USES THEREOF

(71) Applicant: Valero Services, Inc., San Antonio, TX (US)

(72) Inventors: Daryl W. Hanson, San Antonio, TX (US); Javier Quintana, San Antonio, TX (US)

(21) Appl. No.: 17/086,051

(22) Filed: Oct. 30, 2020

(51) Int. Cl. B01D 3/14; B01D 3/00

(52) U.S. Cl. CPC B01D 3/141; B01D 3/32; B01D 3/143; B01D 3/008

(57) ABSTRACT

Enclosed Partition Dividing Wall (EPDW) distillation columns and methods of using EPDW distillation columns are disclosed. The EPDW distillation column includes a column body, a dividing wall, and a wall cap attached to the dividing wall. The wall cap, a portion of column body bound to the wall cap and/or an EPDW surrounding wall, and the dividing wall form an enclosed partition wall rectification section with an opening at the bottom such that a portion of components from the bottom of the column body is separated in the EPDW rectification section.

### CROSS REFERENCE TO RELATED APPLICATIONS

None.

### FIELD OF THE INVENTION

The present invention relates to distillation columns and methods of using distillation columns to separate and/or purify components of a mixture. More specifically, the present invention relates to enclosed partition dividing wall distillation columns that include enclosed rectification section(s) in column bodies, and methods of using enclosed partition dividing wall distillation columns to separate and/or purify components of a mixture.

### BACKGROUND

In petroleum refineries, it is often necessary to separate mixtures of a plurality of components. For example, the separation of a mixture having three components (a ternary mixture) is a common and important process. To ensure the purity of the products, several processes have been developed for separating ternary mixtures in the refining and chemical processing industries.

Conventionally, two columns in series are used for separating three components with the first column separating the lightest component from the other two components. The second column then further separates the other two components from each other. However, in this conventional process, the two heavier components have to be boiled twice, thereby consuming a large amount of energy. Furthermore, the capital expenditure for building two separate columns is relatively high, further increasing the cost of the overall separation process.

Dividing wall distillation columns have also been widely used for separating mixtures of three components. With dividing wall distillation column technology, it is possible to separate a ternary mixture in a single column with a single reboiler. Compared to two columns in series, dividing wall distillation columns may not need to boil the heavier products twice, but there are limitations with this method. For instance, even with dividing wall distillation columns, it can be challenging to separate three components having large differences in flow rates and/or key volatilities to obtain products with acceptable purities.

Overall, while systems and methods for separating a mixture of three components exist, the need for improvements in this field persists in light of at least the aforementioned drawbacks for the conventional systems and methods.

### BRIEF SUMMARY OF THE INVENTION

A solution to at least some of the above mentioned problems associated with systems and methods for separating a mixture of three or more components is discovered. The solution resides in a distillation column that comprises a dividing wall and a wall cap that form an Enclosed Partition Dividing Wall (EPDW) rectification section in a distillation column body that is configured to process a portion of the lower section vapor stream. The EPDW rectification section is configured to restrict components therein from mixing with the components above the wall cap and vice versa. Additionally, the EPDW rectification section is further configured to be operated under different conditions from the other sections of the column. This can be beneficial for separating the mixture that comprises a light key component, a heavy key component, and a small amount of an intermediate component to obtain the intermediate component of high purity, thereby curing the drawback of conventional dividing wall distillation column. It should be noted that the solution described herein can be used with respect to mixtures having more than three components. Additionally, compared to conventional two columns in series, the disclosed enclosed partition dividing wall distillation column does not need to boil bottom components in two columns, thereby reducing energy cost and overall processing cost. Therefore, the enclosed partition dividing wall distillation column of the present invention provides a technical solution to at least some of the problems associated with the conventional systems.

Embodiments of the invention include an Enclosed Partition Dividing Wall (EPDW) distillation column. The Enclosed Partition Dividing Wall distillation column comprises a column body. The EPDW distillation column comprises a dividing wall disposed in the column body so as to (1) divide the column body to form a prefractionation section on a first side of the dividing wall, a bulk fractionation section above a top end of the dividing wall, an EPDW rectification section on a second side of the dividing wall, and a bottom section below a bottom end of the dividing wall; and (2) restrict fluid (liquid and/or gas) flow between the prefractionation section and the EPDW rectification section and vice versa. The EPDW distillation column includes a wall cap restricting fluid flow from the EPDW rectification section to the bulk fractionation section.

Embodiments of the invention include a method of separating a mixture. The method comprises flowing a mixture into a EPDW distillation column. The mixture comprises a first light key component, a first heavy key component, and an intermediate component, wherein the intermediate component comprises less than 15 wt. % of the mixture. The EPDW distillation column comprises a column body. The EPDW distillation column comprises a dividing wall disposed in the column body so as to (1) divide the column body to form a prefractionation section on a first side of the dividing wall, a bulk fractionation section above a top end of the dividing wall, an EPDW rectification section on a second side of the dividing wall, and a bottom section below a bottom end of the dividing wall; and (2) restrict fluid flow from the prefractionation section to the EPDW rectification section. The EPDW distillation column comprises a wall cap restricting fluid flow from the EPDW rectification section to the bulk fractionation section. The EPDW distillation column comprises a first condenser configured to condense vapor exiting the bulk fractionation section to form a top stream and a reflux returning to the bulk fractionation section. The EPDW distillation column comprises a second condenser configured to condense vapor exiting EPDW rectification section to form a side product stream and a second reflux returning to the EPDW rectification section. The method further comprises separating the mixture in the EPDW distillation column to produce the top stream from the bulk fractionation section comprising primarily the first light key component, a bottom stream from the bottom section comprising primarily the heavy key component, and the side product stream from the EPDW rectification section comprising primarily the intermediate component.

Embodiments of the invention include a method of separating a mixture comprising isobutane, n-butane, and alkylate. The method includes flowing the mixture into an EPDW distillation column. The mixture comprises less than 15 wt. % n-butane. The EPDW distillation column includes a column body. The EPDW distillation column includes a dividing wall disposed in the column body so as to (1) divide the column body to form a prefractionation section on a first side of the dividing wall, a bulk fractionation section above a top end of the dividing wall, an EPDW rectification section on a second side of the dividing wall, and a bottom section below a bottom end of the dividing wall; and (2) restrict fluid flow from the prefractionation section to the EPDW rectification section. The EPDW distillation column includes a wall cap restricting fluid flow from the EPDW rectification section to the bulk fractionation section. The method further includes separating the mixture in the EPDW distillation column to produce a top stream from the bulk fractionation section comprising primarily isobutane, a bottom stream from the bottom section comprising primarily alkylate, and a side product stream from the EPDW rectification section comprising primarily n-butane.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or “restricting” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The term “Cₙ+ hydrocarbon” wherein n is a positive integer, e.g. 1, 2, 3, 4, or 5, as that term is used in the specification and/or claims, means any hydrocarbon having at least n number of carbon atom(s) per molecule.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE FIGURES

FIG. 1A shows a schematic diagram of an EPDW distillation column comprising a dividing wall and a wall cap, according to embodiments of the invention;

FIG. 1B shows a cross-sectional view of the EPDW distillation column of FIG. 1A at the location of the wall cap, according to embodiments of the invention;

FIG. 1C shows a perspective view of the EPDW distillation column of FIG. 1A, according to embodiments of the invention;

FIG. 2 shows a schematic diagram of an EPDW distillation column comprising a dividing wall, a wall cap, and EPDW surrounding wall, and method to conduit the vapor stream and reflux stream from and to the EDW rectification section from the distillation tower shell according to embodiments of the invention;

FIG. 3 shows a schematic flowchart of a method of separating a mixture using the EPDW distillation column, according to embodiments of the invention;

FIG. 4 shows a schematic flowchart of a method of separating a mixture of isobutane, n-butane, and alkylate using the EPDW distillation column, according to embodiments of the invention;

FIG. 5 shows key components distribution in EPDW distillation column and in product streams when using EPDW distillation column to separate a mixture comprising n-butane, isobutane, and alkylate.

### DETAILED DESCRIPTION

Currently, a mixture of three components (ternary mixture) is separated using two distillation columns in series, and/or a dividing wall distillation column. However, for two distillation columns in series, the two heavier components in the mixture are boiled twice, resulting in high energy consumption for the separation process. Furthermore, the capital expenditure for the two distillation columns are relatively high compared to single distillation column systems. For conventional dividing wall distillation column systems, it is challenging to separate a mixture that comprises a light component, a heavy component, and a medium component with a concentration significantly lower than the light component and the heavy component, resulting in low product quality and high separation cost. The present invention provides a solution to at least some of these problems. The solution is premised on an Enclosed Partition Dividing Wall (EPDW) distillation column that includes a dividing wall and a wall cap that form an EPDW rectification section in the distillation column. The EPDW distillation column comprises a single column body, thereby avoiding boiling heavier components twice and saving capital expenditure for building the column. Additionally, the EPDW rectification section is configured to be operated under conditions that are different from other sections of the EPDW distillation column, thereby enabling the operating conditions to be optimized for separating the two heavier components of the mixture. Moreover, the EPDW rectification section is configured to restrict mixing of the two heavier components from the EPDW rectification section with the light component in the top section of the EPDW distillation column, resulting in higher purity of the products. These and other non-limiting aspects of the present invention are discussed in further detail in the following sections.

### A. Enclosed Partition Dividing Wall Distillation Column

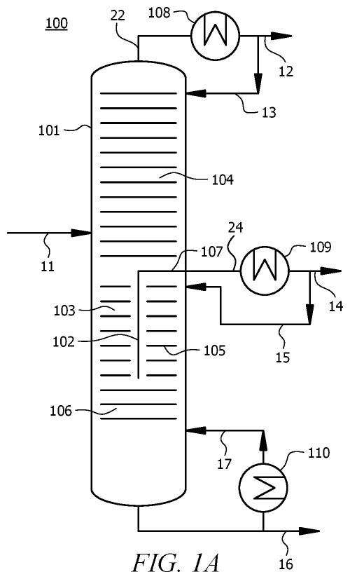

With reference to FIG. 1A, a schematic diagram is shown for an EPDW distillation column 100, which is used for separating mixtures. In embodiments of the invention, the mixtures include three components. Each of the components can include one or more compounds. According to embodiments of the invention, EPDW distillation column 100 includes column body 101. In embodiments of the invention, EPDW distillation column 100 includes column internals comprising trays, plates, packings, or combinations thereof. For instance, EPDW distillation column 100 may comprise valve trays, or sieve trays.

According to embodiments of the invention, EPDW distillation column 100 includes dividing wall 102 disposed in column body 101. In embodiments of the invention, dividing wall 102 is configured to divide column body 101 to form prefractionation section 103 on a first side of dividing wall 102, bulk fractionation section 104 above a top end of dividing wall, EPDW rectification section 105 on a second side of dividing wall 102, and bottom section 106 below a bottom end of dividing wall 102. In embodiments of the invention, dividing wall 102 is further configured to restrict fluid flow from prefractionation section 103 to EPDW rectification section 105. The feed inlet is disposed above or in prefractionation section 103.

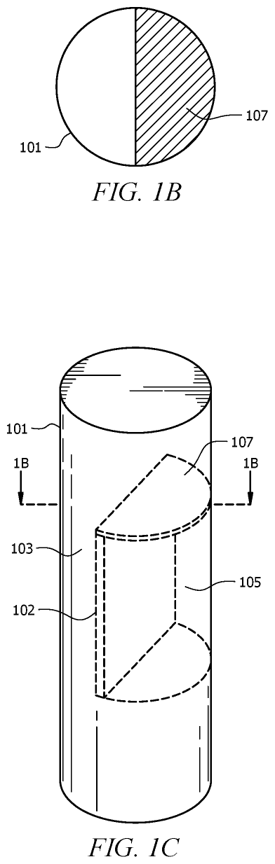

According to embodiments of the invention, EPDW distillation column 100 comprises wall cap 107 configured to restrict fluid flow from EPDW rectification section 105 to bulk fractionation section 104. As shown in FIGS. 1B and 1C, according to embodiments of the invention, the wall cap can be a plate (e.g., horizontal plate or sloped plate) extending over at least a portion of internal cross-sectional area of EPDW distillation column 100. In embodiments of the invention, wall cap 107 is attached to dividing wall 102 so as to form an enclosure of EPDW rectification section with an opening at the bottom thereof. In embodiments of the invention, wall cap 107 is attached to top end of dividing wall 102. In embodiments of the invention, wall cap 107 is in a shape and dimensions such that it is attached to and bound by a portion of column wall of column body 101. In embodiments of the invention, wall cap 107 extends through a portion of internal cross-sectional area of EPDW distillation column 100 and forms EPDW rectification section 105 with dividing wall 102 and EPDW surrounding wall 115, as shown in FIG. 2. In embodiments of the invention, as shown in FIG. 2, ducting and/or piping of second condenser 109 and the reflux of vapor exiting EPDW rectification section 105 are connected directly to EPDW rectification section 105. Dividing wall 102, in embodiments of the invention, can be vertical or sloped, and wall cap 107 can be horizontal or sloped. In embodiments of the invention, wall cap 107 can have various shapes, for example, a shape determined by the shape of column body 101. In embodiments of the invention, wall cap 107 call be can have a round shape, a square shape, a polygon shape, a chordal shape, or any irregular shape.

According to embodiments of the invention, distillation column 100 comprises first condenser 108 configured to condense vapor exiting bulk fractionation section 104 to form top stream 12 and top reflux stream 13 returning to bulk fractionation section 104. In embodiments of the invention, the vapor exiting bulk fractionation section 104 are completely or substantially condensed. In embodiments of the invention, the vapor exits bulk fractionation section 104 at a top portion of column body 101. According to embodiments of the invention, distillation column 100 comprises second condenser 109 configured to condense vapor exiting EPDW rectification section 105 to form side product stream 14 and middle reflux stream 15 returning to EPDW rectification section 105. In embodiments of the invention, side vapor stream 24 exiting EPDW rectification section is completely condensed or substantially condensed. In embodiments of the invention, top vapor stream 22 exits EPDW rectification section 105 between wall cap 107 and the top fractioning element (tray or packing element) of EPDW rectification section 105. In embodiments of the invention, the top fractioning element includes a piece of equipment that is configured to fractionate component and/or aid in fractionation efficiency (e.g., for mist elimination, etc.). In embodiments of the invention, first condenser 108 and second condenser 109 can be operated independently. First condenser 108 can be operated under different condensing temperatures and/or different condensing pressure from second condenser 109. In embodiments of the invention, first condenser 108 and second condenser 109 can be controlled by different flow rate controllers and/or different pumps. According to embodiments of the invention, EPDW distillation column 100 comprises reboiler 110 configured to boil bottom components exiting bottom section 106 of column body 101 to produce bottom stream 16 and reboiler vapor stream 17 returning to bottom section 106. In embodiments of the invention, a feed inlet is disposed on column body 101 configured to receive feed stream 11 therein.

In embodiments of the invention, dividing wall 102 starts at 63 to 68% of total theoretical tray number (from top) of EPDW distillation column 100, and ends at 79 to 85% of total theoretical tray number of EPDW distillation column 100. The feed inlet may be disposed at 20 to 25% of total theoretical tray number of EPDW distillation column 100. In embodiments of the invention, EPDW distillation column 100 can include two or more EPDW dividing walls and wall caps forming two or more EPDW rectification sections. Different EPDW rectification sections can be of different heights and may be at different vertical and horizontal locations within EPDW distillation column 100.

### B. Method of Separating a Mixture

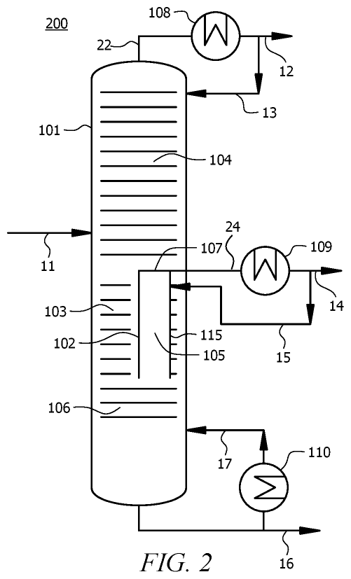

Methods of separating a mixture have been discovered. As shown in FIG. 3, embodiments of the invention include method 300 for separating a mixture comprising a first light key component, a heavy key component, and an intermediate component. Method 300 may be implemented by EPDW distillation column 100. According to embodiments of the invention, as shown in block 301, method 300 includes flowing a mixture of feed stream 11 into EPDW distillation column 100. The mixture can comprise a first light key component, a heavy key component, and an intermediate component. The intermediate component may comprise less than 15 wt. %, preferably less than 5 wt. %, of the mixture. In embodiments of the invention, the mixture can further include components lighter than the first light key component, and/or components heavier than the heavy key component.

According to embodiments of the invention, as shown in block 302, method 300 includes separating the mixture in EPDW distillation column to produce top stream 12 comprising primarily the first light key component and/or components lighter than first key component, bottom stream 16 comprising primarily the heavy key component and components heavier than heavy key component, and side product stream 14 comprising primarily the intermediate component. Side product stream 14 may comprise 95.0 to 99.9 wt. % the intermediate component and all ranges and values there between including ranges of 95.0 to 95.5 wt. %, 95.5 to 96.0 wt. %, 96.0 to 96.5 wt. %, 96.5 to 97 wt. %, 97 to 97.5 wt. %, 97.5 to 98.0 wt. %, 98.0 to 98.5 wt. %, 98.5 to 99.0 wt. %, 99.0 to 99.5 wt. %, and 99.5 to 99.9 wt. %.

i) Method of Separating a Mixture Comprising Isobutane, n-Butane, and Alkylate

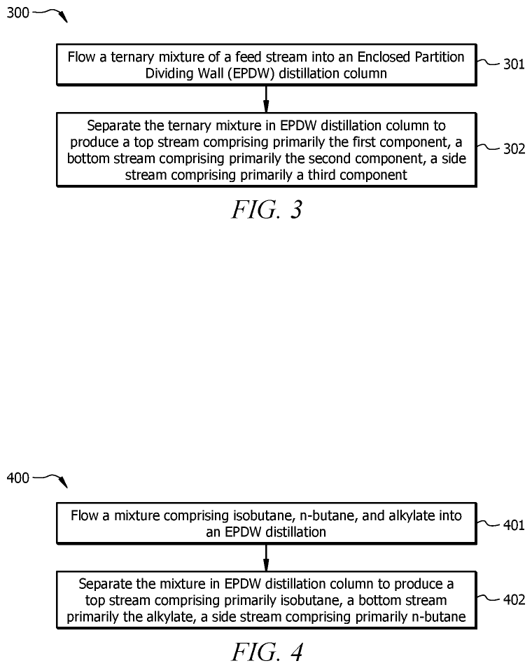

Methods of separating a mixture comprising primarily isobutane, n-butane, and alkylate are discovered. As shown in FIG. 4, embodiments of the invention include method 400 for separating a mixture comprising isobutane, n-butane, and alkylate. Method 400 may be implemented by EPDW distillation column 100 as shown in FIG. 1A.

According to embodiments of the invention, as shown in 401, method 400 includes flowing the mixture comprising isobutane, n-butane, and alkylate into EPDW distillation column 100. In embodiments of the invention, the mixture may further include one or more compounds lighter than isobutane. In embodiments of the invention, the mixture comprises less than 15 wt. % n-butane and all ranges and values there between including ranges of 0.01 to 3 wt. % n-butane, 3 to 6 wt. % n-butane, 6 to 9 wt. % n-butane, 9 to 12 wt. % n-butane, and 12 to 15 wt. % n-butane. In embodiments of the invention, the mixture can further comprise C₅+ hydrocarbons.

In embodiments of the invention, for method 400, dividing wall 102 starts from 63 to 68% of total theoretical tray number and ends at 79 to 85% of theoretical tray number. In embodiments of the invention, for method 400, the inlet of EPDW distillation column 100 is disposed between 20 to 30% of total theoretical tray number and all ranges and values there between including ranges of 20 to 21%, 21 to 22%, 22 to 23%, 23 to 24%, 24 to 25%, 25 to 26%, 26 to 27%, 27 to 28%, 28 to 29%, and 29 to 30%.

According to embodiments of the invention, as shown in block 402, method 400 includes separating the mixture in EPDW distillation column 100 to produce top stream 12 from bulk fractionation section 104 comprising primarily isobutane, bottom stream 16 from bottom section 106 comprising primarily the alkylate, and side product stream 14 from EPDW rectification section 105 comprising primarily n-butane. In embodiments of the invention, at block 402, first condenser 108 and second condenser 109 are operated independently. In embodiments of the invention, at block 402, EPDW rectification section 105 is operated under different operating temperatures and/or pressures from prefractionational section 104, bottom section 106, and/or bulk fractionation section 104. In embodiments of the invention, EPDW rectification section 105 is operated with different composition gradients from prefractionational section 104, bottom section 106, and/or bulk fractionation section 104.

In embodiments of the invention, at block 402, EPDW rectification section 105 is operated at a condensing temperature of 60 to 88° C. and all ranges and values there between including ranges of 60 to 62° C., 62 to 64° C., 64 to 66° C., 66 to 68° C., 68 to 70° C., 70 to 72° C., 72 to 74° C., 74 to 76° C., 76 to 78° C., 78 to 80° C., 80 to 82° C., 82 to 84° C., 84 to 86° C., and 86 to 88° C. EPDW rectification section 105, at block 402, may be operated at an operating pressure of 80 to 90 psig and all ranges and values there between including ranges of 80 to 82 psig, 82 to 84 psig, 84 to 86 psig, 86 to 88 psig, and 88 to 90 psig.

In embodiments of the invention, bulk fractionation section 104 is operated in a temperature range of 49 to 74° C. and all ranges and values there between including ranges of 49 to 54° C., 54 to 59° C., 59 to 64° C., 64 to 69° C., and 69 to 74° C. At block 402, bottom section 106 can be operated in a temperature range of 90 to 113° C. and all ranges and values there between including ranges of 90 to 93° C., 93 to 96° C., 96 to 99° C., 99 to 102° C., 102 to 105° C., 105 to 108° C., 108 to 111° C., and 111 to 113° C. According to embodiments of the invention, at block 402, prefractionation section is operated at a temperature in a range of 73 to 91° C. and all ranges and values there between including ranges of 73 to 75° C., 75 to 77° C., 77 to 79° C., 79 to 81° C., 81 to 83° C., 83 to 85° C., 85 to 87° C., 87 to 89° C., and 89 to 91° C. In embodiments of the invention, at block 402, bulk fractionation section 104, prefractionation section 103, and/or bottom section 106 are operated at an operating pressure of 75 to 90 psig and all ranges and values there between including ranges of 75 to 78 psig, 78 to 81 psig, 81 to 84 psig, 84 to 87 psig, and 87 to 90 psig. According to embodiments of the invention, side vapor stream 24 is drawn 105 between wall cap 107 and the top fractioning element (tray or packing element) of EPDW rectification section 105, and/or a location in close proximity to the entrance where middle reflux stream 15 returns to EPDW rectification section 105. In embodiments of the invention, the top fractioning element includes a piece of equipment that is configured to fractionate component and/or aid in fractionation efficiency (e.g., for mist elimination, etc.). According to embodiments of the invention, side vapor stream 24 is drawn from above to a top tray of EPDW rectification section 105, and/or a location in close proximity to the entrance where the EPDW rectification reflux stream 15 returns to EPDW rectification section 105. In embodiments of the invention, side vapor stream 24 is totally or substantially condensed to form side product stream 14 and the EPDW rectification reflux stream 15.

In embodiments of the invention, at block 402, side product stream 14 comprises 98 to 99.9 wt. % n-butane, and all ranges and values there between including ranges of 98 to 98.2 wt. %, 98.2 to 98.4 wt. %, 98.4 to 98.6 wt. %, 98.6 to 98.8 wt. %, 98.8 to 99.0 wt. %, 99.0 to 99.2 wt. %, 99.2 to 99.4 wt. %, 99.4 to 99.6 wt. %, 99.6 to 99.8 wt. %, and 99.8 to 99.9 wt. %. In embodiments of the invention, at block 402, top stream 12 comprises 90 to 97 wt. % isobutane and all ranges and values there between including ranges of 90 to 91 wt. %, 91 to 92 wt. %, 92 to 93 wt. %, 93 to 94 wt. %, 94 to 95 wt. %, 95 to 96 wt. %, and 96 to 97 wt. %. At block 402, bottom stream 16 may comprise 96 to 99.9 wt. % alkylate and all ranges and values there between including ranges of 96 to 96.5 wt. %, 96.5 to 97 wt. %, 97 to 97.5 wt. %, 97.5 to 98 wt. %, 98 to 98.5 wt. %, 98.5 to 99.0 wt. %, 99.0 to 99.5 wt. %, and 99.5 to 99.9 wt. %.

The systems and processes described herein can also include various equipment that is not shown and is known to one of skill in the art of chemical processing. For example, some controllers, piping, computers, valves, pumps, heaters, thermocouples, pressure indicators, mixers, heat exchangers, and the like may not be shown.

As part of the disclosure of the present invention, specific examples are included below. The examples are for illustrative purposes only and are not intended to limit the invention. Those of ordinary skill in the art will readily recognize parameters that can be changed or modified to yield essentially the same results.

### Example

### Separation of n-Butane, Isobutane, and Alkylate Using an EPDW Distillation Column

Simulations were run for separation of mixture comprising ethane, propane, isobutane, butane, isopentane, n-pentane, and C₆ to C₈ hydrocarbons in an EPDW distillation column as shown in FIG. 1. The mixture was fed into the EPDW distillation column at a flow rate of 294,835 kg/hr. The EPDW distillation column included 85 trays total. The feed inlet was at 18^{th} tray from the top. The dividing wall was disposed from the 57^{th} tray to the 70^{th} tray. The bulk fractionation section was from the 1^{st} tray to the 70^{th} tray. The bottom section was from the 71^{st} tray to the 85^{th} tray. The EPDW distillation column was operated at a top pressure of 80 psig. The bulk fractionation section was operated at a temperature of 49 to 74° C., the bottom section was operated in a temperature range of 90 to 113° C., and the prefractionation section was operated in a temperature range of 73 to 91° C.

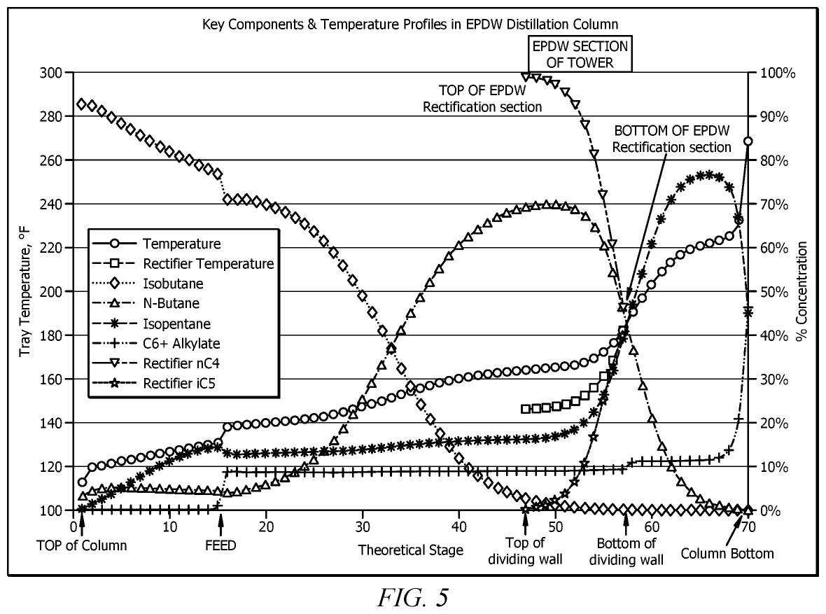

The results of components distribution are shown in FIG. 5. Overall, the top stream from the EPDW distillation column included about 92.7 wt. % isobutane, and about 0.5 wt. % isopentane. The bottom stream from the EPDW distillation column included 99.86 wt. % C₅+ hydrocarbons. The side product stream from the EPDW distillation column included about 99.6 wt. % n-butane.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. An Enclosed Partition Dividing Wall (EPDW) distillation column comprising:
a column body;
a dividing wall disposed in the column body so as to (1) divide the column body to form a prefractionation section on a first side of the dividing wall, a bulk fractionation section above a top end of the dividing wall, an Enclosed Partition Dividing Wall (EPDW) rectification section on a second side of the dividing wall, and a bottom section below a bottom end of the dividing wall, and (2) restrict fluid flow between the prefractionation section and the EPDW rectification section; and
a wall cap restricting fluid flow from the EPDW rectification section to the bulk fractionation section.

2. The EPDW distillation column of claim 1, further comprising:
a first condenser configured to condense vapor exiting the bulk fractionation section to form a top stream and a reflux returning to the bulk fractionation section; and
a second condenser configured to condense vapor exiting the EPDW rectification section to form a side product stream and the reflux returning to EPDW rectification section.

3. The EPDW distillation column of claim 1, wherein the prefractionation section and the EPDW rectification section are configured to be operated under different operating conditions.

4. The EPDW distillation column of claim 1, wherein the EPDW distillation column is configured to separate a mixture that includes a minor component comprising less than 15 wt. % of the mixture.

5. The EPDW distillation column of claim 4, wherein the minor component is recovered in a side product stream from the EPDW rectification section of the distillation column.

6. The EPDW distillation column of claim 1, wherein the EPDW distillation column comprises two or more EPDW rectification sections.

7. A method of separating a mixture, the method comprising:
flowing a mixture into an Enclosed Partition Dividing Wall (EPDW) distillation column, wherein the mixture comprises a first light key component, a heavy key component, and an intermediate component, and the intermediate component comprises less than 15 wt. % of the mixture, wherein the EPDW distillation column comprises:
a column body;
a dividing wall disposed in the column body so as to (1) divide the column body to form a prefractionation section on a first side of the dividing wall, a bulk fractionation section above top end of the dividing wall, an EPDW rectification section on a second side of the dividing wall, and a bottom section below a bottom end of the dividing wall, and (2) restrict fluid flow between the prefractionation section and the EPDW rectification section;
a wall cap restricting vertical fluid flow from the EPDW rectification section to the bulk fractionation section;
a first condenser configured to condense vapor exiting the bulk fractionation section to form a top stream and a reflux returning to the bulk fractionation section;
a second condenser configured to condense vapor exiting the EPDW rectification section to form a side product stream and a reflux returning to EPDW rectification section; and
separating the mixture in the EPDW distillation column to produce the top stream from the bulk fractionation section comprising the first light key component and components lighter than first light key component, a bottom stream from the bottom section comprising the heavy key component and components heavier than the heavy key component, and the side product stream from the EPDW rectification section comprising primarily the intermediate component;
wherein the EPDW rectification section is operated under different operating temperatures and pressures from the prefractionation section and the bottom section.

8. The method of claim 7, wherein the first condenser and the second condenser are operated under different conditions.

9. (canceled)

10. The method of claim 7, wherein boiling point difference between the first light key component and the intermediate component is less than 27.8° C., and the boiling point difference between the heavy key component and the intermediate component is less than 64° C.

11. The method of claim 7, wherein, in the separating step, the EPDW section contains primarily the heavy key component and the intermediate component, collectively.

12. The method of claim 11, wherein the EPDW section is operated under operating conditions sufficient to separate the heavy key component and the intermediate component.

13. A method of separating a mixture comprising isobutane, n-butane, and alkylate, the method comprising:
flowing the mixture into an Enclosed Partition Dividing Wall (EPDW) distillation column, wherein the mixture comprises less than 15 wt. % n-butane, and wherein the EPDW distillation column comprises:
a column body;
a dividing wall disposed in the column body so as to (1) divide the column body to form a prefractionation section on a first side of the dividing wall, a bulk fractionation section above top end of the dividing wall, an EPDW rectification section on a second side of the dividing wall, and a bottom section below a bottom end of the dividing wall, and (2) restrict fluid flow between the prefractionation section and the EPDW rectification section; and
a wall cap restricting fluid flow from the EPDW rectification section to the bulk fractionation section; and
separating the mixture in the EPDW distillation column to produce a top stream from the bulk fractionation section comprising primarily the isobutane, a bottom stream from the bottom section comprising primarily the alkylate, and a side product stream from the EPDW rectification section comprising primarily the n-butane;
wherein the EPDW rectification section is operated under different operating temperatures and pressures from the prefractionation section and the bottom section.

14. The method of claim 13, wherein the EPDW distillation column further comprises:
a first condenser configured to condense vapor exiting the bulk fractionation section to form the top stream and a reflux returning to the bulk fractionation section; and
a second condenser configured to condense vapor exiting EPDW rectification section to form the side product stream and the reflux returning to EPDW rectification section.

15. The method of claim 14, wherein the first condenser and the second condenser are operated independently.

16. The method of claim 13, wherein the EPDW rectification section is operated under different operating temperatures and pressures from the prefractionation section and the bottom section, and wherein EPDW rectification section is operated with different composition gradients from the prefractionation section and the bottom section.

17. The method of claim 16, wherein the EPDW rectification section is operated at a condensing temperature of 60 to 88° C. and an operating pressure of 80 to 90 psig.

18. The method of claim 17, wherein the bulk fractionation section is operated in a temperature range of 49 to 74° C., the bottom section is operated in a temperature range of 90 to 113° C., and the prefractionation section is operated in a temperature range of 73 to 91° C.

19. The method of claim 17, wherein remaining sections other than the EPDW rectification section are operated at an operating pressure of 75 to 90 psig.

20. The method of claim 13, wherein the side product stream comprises 98 to 99.9 wt. % n-butane.

21. The method of claim 13, wherein the top stream comprises 90 to 97 wt. % isobutane.

22. The method of claim 13, wherein the bottom stream comprises 95 to 99.9 wt. % alkylate.

23. The method of claim 13, wherein the side product stream is drawn between the wall cap and uppermost tray or packing internal inside the EPDW rectification section and/or a location in a close proximity to reflux return entrance of the EPDW rectification section.

24. The method of claim 13, wherein the dividing wall of the enclosed partition dividing wall distillation column starts at 63 to 68% of total theoretical tray number of the enclosed partition dividing wall distillation column, counted from the top to the bottom, and ends at 79 to 85% of total theoretical tray number of the enclosed partition dividing wall distillation column, counted from the top to the bottom.

25. The method of claim 13, wherein the mixture is flowed into the dividing wall distillation column at 20 to 30% of total theoretical tray number of the EPDW distillation column, counted from the top to the bottom.
